(19) United States
(12) Patent Application Publication
(10) Pub. No.: US 2012/0041515 A1
(43) Pub. Date: Feb. 16, 2012

(54) WIRELESS REMOTE DEVICE FOR A HEARING PROSTHESIS

(76) Inventors: Werner Meskens, Opwijk (BE); Jan Janssen, St. Ives (AU); Peter Gibson, South Coogee (AU); Tony Nygard, Terrigal (AU); Mathew Markey, Oatley (AU)

(21) Appl. No.: 12/857,398

(22) Filed: Aug. 16, 2010

(51) Int. Cl. A61F 11/04; A61N 1/36

(52) U.S. Cl. 607/57

(57) ABSTRACT

A hearing prosthesis and method for same are disclosed wherein a smaller wireless microphone component transmits audio signals to an external component of the medical device. The external component processes the received audio signal(s) to generate stimulation data. The external component is detachably connected to a coil that transmits power and the stimulation data, via magnetic induction, to an implanted component. The implanted component applies stimulation to a recipient in accordance with the received stimulation data.

### BACKGROUND

1. Field of the Invention

The present invention relates generally to an implantable medical device, and more particularly, to a hearing prosthesis comprising a component for wirelessly transmitting received sound.

2. Related Art

Medical devices having one or more implantable components, generally referred to as implantable medical devices, have provided a wide range of therapeutic benefits to patients over recent decades. In particular, implantable medical devices such as hearing prostheses, pacemakers, defibrillators, functional electrical stimulation devices, organ assist or replacement devices, and other partially- or completely-implanted medical devices have been successful in performing life saving and/or lifestyle enhancement functions for a number of years.

A variety of implantable medical devices have been developed to deliver controlled electrical stimulation to a region of a patient's body. One such device which provides hearing sensation to individuals with sensorineural hearing loss is the implantable hearing prostheses. Exemplary implantable hearing prostheses include, for example, cochlear implants, auditory brainstem implants (ABIs), and middle ear implants.

For individuals with sensorineural hearing loss, there is typically damage to or an absence of hair cells within the cochlea which convert sound into nerve impulses which are perceived as sound by the brain. Unfortunately, such individuals are unable to derive suitable benefit from acoustic hearing aids, and hence look to rely upon cochlear implants to provide them with the ability to perceive sound.

Cochlear implants use electrical stimulation of auditory nerve cells to bypass absent or defective hair cells that normally transduce acoustic vibrations into neural activity. Such devices generally use an array of electrode contacts implanted into the scala tympani of the cochlea to deliver stimulation that differentially activates auditory neurons that normally encode differential frequencies of sound. As used herein the term cochlear implant includes hearing prostheses that deliver electrical stimulation in combination with other types of stimulation, such as acoustic or mechanical stimulation.

Auditory brain implants are used to treat a smaller number of recipients with bilateral degeneration of the auditory nerve. For such recipients, the auditory brain implants provides stimulation of the cochlear nucleus in the brainstem. Auditory brain implants similarly use a plurality of electrode contacts to provide stimulation to the recipient.

Hearing prostheses typically use an external component to process received sound and generate corresponding stimulation data specifying the stimulation to be applied to the recipient by the implanted component. This external component is typically large in size. In adults this external component is often a behind-the-ear (BTE) device that includes a microphone for receiving the sound. In children, however, the external component may be too large to fit behind the child's ear.

### SUMMARY

In one aspect of the present invention there is provided a hearing prosthesis comprising an implantable component configured to deliver stimulation to a recipient of the hearing prosthesis system, a first smaller component, a second larger component, and a first coil system. The first smaller external component comprises at least one sound input element configured to be located on, near, or in a recipient's ear and configured to receive sound and generate an electronic signal based on the received sound; and a first wireless interface configured to wirelessly transmit the electronic signal. The second larger external component comprises a second wireless interface configured to receive the transmitted electronic signal; a processor configured to process the received electronic signal and generate corresponding stimulation data that specifies stimulation; a coil interface configured to transfer the stimulation data; a power supply; and a user interface. The first coil system comprises a first coil, a cable and a connector, wherein the connector is detachably connectable to the coil interface of the second external component, wherein the cable is configured to enable the second external component to be positioned, when the cable connects the second external component and the first coil, at a location remote from the first external component, and wherein the first coil is configured to transcutaneously transfer the stimulation data to the implantable component.

In another aspect, there is provided a method comprising: receiving sound at a first external component; generating an electronic signal based on the received sound; wirelessly transmitting the electronic signal from the first external component to a second external component, where the first external component has a smaller size than the second external component and is located on, near, or in a recipient's ear; transferring data corresponding to the received sound to an internal component transcutaneouly over a wireless link; and applying stimulation to a recipient in accordance with the transmitted data.

In yet another embodiment, there is provided a hearing prosthesis system comprising: means for receiving sound at a first external component; means for generating an electronic signal based on the received sound; means for wirelessly transmitting the electronic signal from the first external component to a second external component, where the first external component has a smaller size than the second external component and is configured to be located on, near or in a recipient's ear; means for transferring data corresponding to the received sound to an internal component transcutaneouly over a wireless link; means for detachably connecting the second external component to a cable configured to connecting the second external component and the means for transferring, wherein the cable is configured to enable the second external component to be positioned, when the cable connects the second external component and the means for transferring, at a location remote from the first external component; means for transcutaneously receiving the stimulation data; and means for applying stimulation to a recipient in accordance with the stimulation data.

### BRIEF DESCRIPTION OF THE DRAWINGS

Embodiments of the present invention are described below with reference to the attached drawings, in which:

FIG. 1A is a perspective view of an exemplary cochlear implant, in accordance with an embodiment of the invention;

FIG. 1B is a perspective view of an exemplary cochlear implant, in accordance with an embodiment of the invention;

FIG. 2 is a functional block diagram of the cochlear implant of FIG. 1A, in accordance with an embodiment of the invention;

FIG. 3 illustrates an alternative embodiment of a cochlear implant, in accordance with an embodiment of the present invention;

FIG. 4 illustrates an embodiment in which the first external component comprises a primary coil interface similar to the primary coil interface of the second external component, in accordance with an embodiment of the present invention;

FIG. 5 illustrates an embodiment in which a second external component and a first external component both communicate wirelessly with an internal component, in accordance with an embodiment of the present invention;

FIG. 6 illustrates a cochlear implant comprising first and second external components and an internal component, where the first external component supports part of the weight of the cable connected to the second external component, in accordance with an embodiment of the invention;

FIG. 7 provides a simplified flow chart for receiving sound and providing corresponding stimulation, in accordance with an embodiment of the present invention; and

FIG. 8 illustrates an alternative embodiment of a cochlear implant, in accordance with an embodiment of the present invention.

### DETAILED DESCRIPTION

Embodiments of the present invention are generally directed to a hearing prosthesis configured to apply stimulation in accordance with received sound. As will be discussed in more detail below, in an embodiment, a wireless microphone component that wirelessly transmits electronic signals representing the received sound to a relatively larger sound processor component. In embodiments, the wireless microphone component may be configured as, for example, an in the ear device (ITE), an in the canal (ITC) device, a completely in canal (CIC) device, a mini-Behind the Ear (BTE) device, or a micro-BTE device.

The sound processor component processes the received signals and generates corresponding stimulation data. This stimulation data specifies the stimulation to be applied by the hearing prosthesis. In embodiments, the sound processor component may be, for example, a wireless remote control device or an adult size BTE. The sound processor component may be detachably connected to a coil for transmitting the stimulation data and power via magnetic induction to an internal component. The internal component is configured to receive the stimulation data and power from the sound processor component and apply stimulation to the recipient in accordance with the received stimulation data.

Use of an embodiment, such as noted above, may be beneficial in implementations in which the hearing prosthesis is fitted to a child. In such implementations, that sound processor component may be too large to fit in, on, or near the ear of the child. The sound processor component may thus be placed away from the ear of the child, such as, for example, in a pouch on the shoulder or back of the child. If a microphone included in the sound processor component is used this may result in spatial issues for the child due to sound arriving from behind the child sounding as though it is arriving from the side of the child. In embodiments, a small wireless microphone component, such as noted above, may be positioned in, on, or near child's error. The sound received by this wireless microphone component may be converted to an electronic signal and wirelessly transmitted to the sound processor component, which generates the stimulation data.

Embodiments of the present invention are described herein primarily in connection with one type of implantable hearing prosthesis, namely a cochlear prosthesis (commonly referred to as cochlear prosthetic devices, cochlear implants, cochlear devices, and the like; simply “cochlea implants” herein.) Cochlear implants deliver electrical stimulation to the cochlea of a recipient. It should, however, be understood that the techniques described herein are also applicable to other types of hearing prosthesis, such as, auditory brain stimulators, also sometimes referred to as an auditory brainstem implant (ABI), and electro-mechanical stimulation implants (e.g., direct acoustic cochlear stimulators (DACS) and transcutaneous BAHA (T-BAHA) implants).

As used herein, cochlear implants also include hearing prostheses that deliver electrical stimulation in combination with other types of stimulation, such as acoustic or mechanical stimulation (sometimes referred to as mixed-mode devices). It would be appreciated that embodiments of the present invention may be implemented in any cochlear implant or other hearing prosthesis now known or later developed, including auditory brain stimulators, or implantable hearing prostheses that mechanically stimulate components of the recipient's middle or inner ear. For example, embodiments of the present invention may be implemented, for example, in a hearing prosthesis that provides mechanical stimulation to the middle ear and/or inner ear of a recipient.

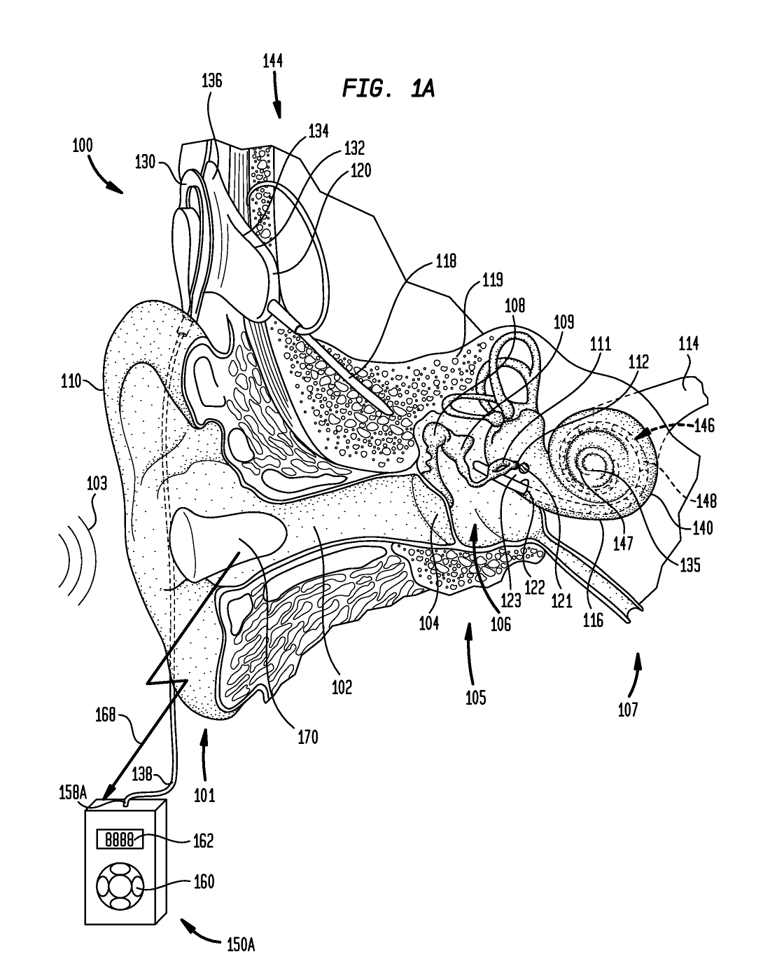

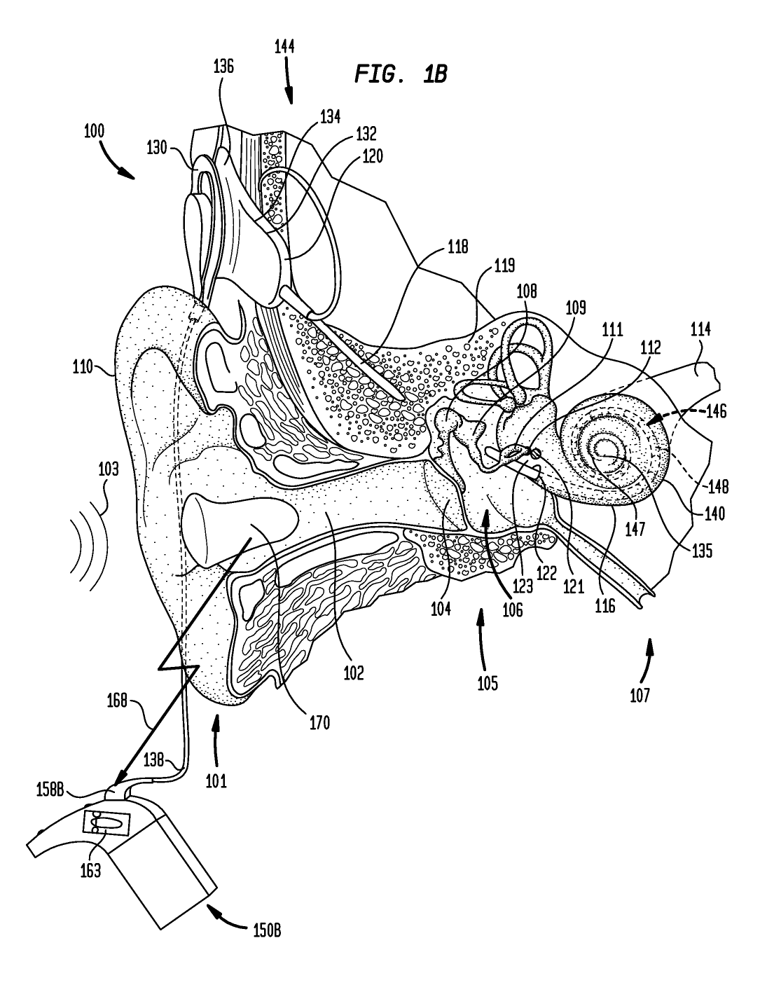

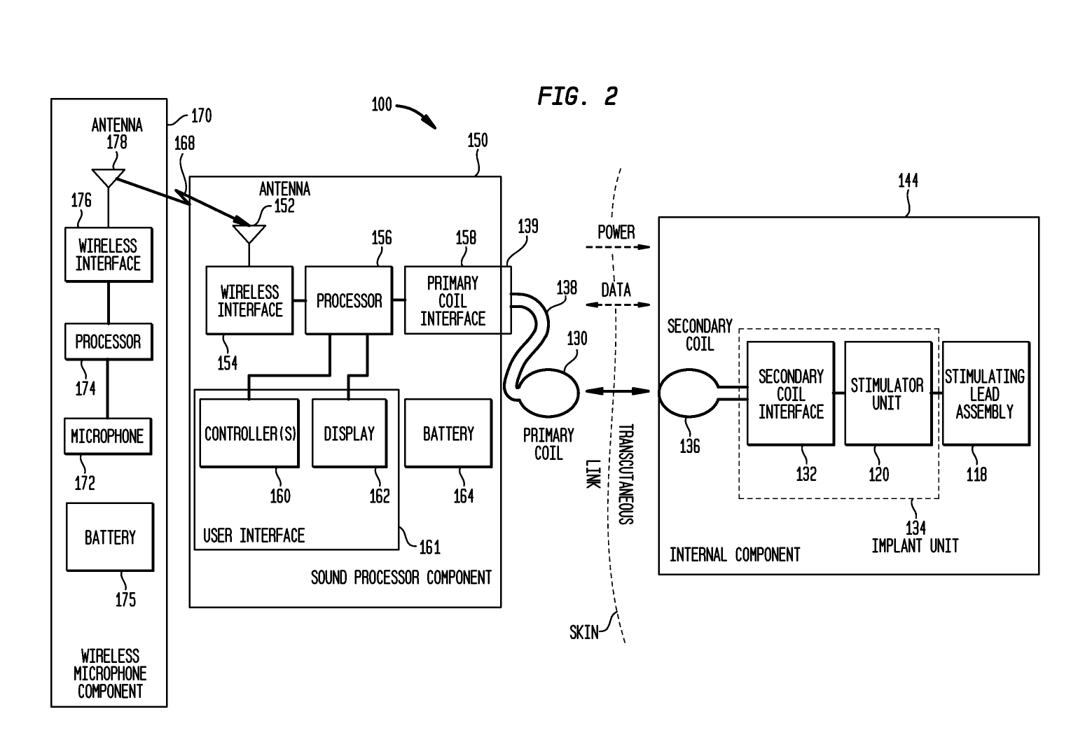

FIG. 1A is perspective view of a cochlear implant, referred to as cochlear implant 100 implanted in a recipient. FIG. 2 is a functional block diagram of cochlear implant 100. The recipient has an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph (not shown) within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

Cochlear implant 100 comprises a first external component 170, a second external component 150A, a primary coil 130, and an internal component 144. In the illustrated embodiment, first external component 170 is a small wireless microphone component. For ease of explanation, first external component 170 will hereinafter be referred to a wireless microphone component 170 and second external component 150A referred to as a sound processor component 150A.

In embodiments, wireless microphone component 170 may be configured for implantation in the ear canal 102 of the recipient (e.g., the ear canal of a child). Or, for example, wireless microphone component 170 may be configured for attaching to a pram (also referred to as a stroller), a hat, clothing, or other object. In the presently discussed embodiment, wireless microphone component 170 is configured to have a smaller size than that of second external component 150A.

Because the illustrated wireless microphone component 170 is configured for placement in the recipient's ear, it is referred to as an in the ear (ITE) device. In other embodiments, the wireless microphone component 170 may be configured differently. For example, wireless microphone component 170 may be configured as a in the canal (ITC) device, a completely in canal (CIC) device, or a Behind the Ear (BTE) device. An ITE device refers to a device that partially or fully fills the outer ear of the recipient. ITE devices are often custom made for the recipient. An ITC device refers to a device that is configured to fit in the recipient's ear canal. ITC devices are typically slightly smaller than ITE devices, but larger than CIC devices. A CIC device refers to a device configured to fit deep within the ear canal of the recipient so that it is not readily visible to an observer. A BTE device is a device configured to fit behind the ear of the recipient. For example, in an embodiment, the wireless microphone component 170 may be a mini BTE or a micro BTE configured to fit behind the ear of the recipient. A mini BTE is a device similar to the a standard BTE, but with a smaller replaceable battery, such that the overall dimensions of a mini BTE are smaller than that of a standard BTE. A micro BTE is an even smaller BTE device.

As illustrated, wireless microphone component 170 comprises one or more sound input elements 172 (e.g., microphone(s)), a processor 174, a power supply (e.g., a battery) 175, a wireless interface 176, and an antenna 178. In operation, sound 103 is received by microphone 172, which converts the sound to electronic signals. The electronic sounds are provided to processor 174, which performs any appropriate processing of the received sound, such as, for example, filtering, equalization etc. Or, for example, processor 174 may include processing capabilities similar to processor 156 of the second external device 150A, which will be discussed in more detail below. Or, in other embodiments, processor 174 need not be used and the electronic signal from microphone 172 is provided directly to wireless interface 176. Wireless interface 176 converts the processed electronic signals to radio frequency (RF), such as for example a frequency of 2.4 GHz and transmits the electronic signal via antenna 178 to the second external component over RF link 168.

Power supply 175 provides power for operations of the wireless microphone component 170. Power supply 175 may be, for example, a rechargeable battery that is either permanently installed in wireless microphone component 170 or may be removable from second external component 170. Or, for example, the battery may be a non-rechargeable replaceable battery.

As illustrated, second external component 150A, also referred to herein as sound processor component 150A, comprises an antenna 152, a wireless interface 154, a processor 156, an external coil driver unit 158A (referred to herein as primary coil interface 158), one or more controllers 160, a display 162, and a power source 164. External coil interface unit 158A is configured to detachably connect second external component 150A to an external coil 130 (also referred to herein as primary coil 130) via a cable 138 comprising a connector 139 configured to detachably connect to primary coil interface 158.

Primary coil 130 may contain a magnet (not shown) that may be secured directly or indirectly concentric to internal coil 136 (also referred to herein as secondary coil 136). External and internal coils are closely coupled enabling power and data transfers by an inductive link. Although not illustrated, in embodiments, second external component 150A may also comprise one or more sound input elements, such as microphone 124 for detecting sound.

As illustrated, the RF electronic signal transmitted by wireless microphone component 170 is received by antenna 152 of second external component 150A. Antenna 152 provides the received electronic signal 154 to wireless interface 154, which may demodulate the received electronic signal and provide the demodulated electronic signal to processor 156. Processor 156 processes the received electronic signal and generates stimulation data specifying the stimulation to be applied by cochlear implant 100. This stimulation data may be encoded to generate encoded signals, sometimes referred to herein as encoded data signals, which are provided to the external coil interface unit 158A (also referred to herein as “primary coil interface 158A”) via a cable 138.

The sound processor component 150A may be configured for providing power and/or data to the internal component 144 of the cochlear implant 100. In the illustrated embodiment, the sound processor component 150A may be configured for use with a child. For example, the sound processor component 150A may be configured for placement in a pouch (e.g., a pocket) included in the clothing of the child (e.g., on the back or shoulder of the child). Sound processor component 150A may include one more fastening devices (e.g., clips) for helping retain the sound processor component 150A in the pouch or wherever the sound processor component 150A is to be placed.

The cable 138 may be a relatively long cable configured for connecting the primary coil 130 to the sound processor component 150A when placed in a pouch located on the back or shoulder of the recipient, or for, example, near a pocket in the pants of the recipient. In embodiments described herein, cable 138 may be referred to as an extension cable, and may be, for example, 20 to 100 cm long.

As noted, primary coil interface 158A may configured for detachably connecting the sound processor component 150A to cable 138 via a connector 139. Further, the primary coil 130 may similarly include an interface (e.g., a connector) for detachably connecting the primary coil 130 to cable 138. The combination of the cable 138, connector 139, and primary coil interface 158 may be collectively referred to as a connector system.

Primary coil interface 158A may further comprise the coil drivers for driving primary coil 130 in transcutaneously transmitting data via magnetic induction. Or, for example, in another embodiment, the coil drivers for driving coil 130 may be included in the or near the primary coil 130. For example, the primary coil 130 may be included in a printed circuit board (PCB) coated in a plastic housing. In an embodiment, the PCB may also comprise the coil drivers for driving primary coil 130. Such an embodiment may be useful in embodiments where the cable 138 length is long.

In the illustrated embodiment of FIG. 1A, the sound processor component 150A is configured as a remote control unit for controlling certain operation of cochlear implant 100 and/or receiving data regarding the operations (e.g., the stimulation rate, battery life, etc.) of the cochlear implant 100. As illustrated, sound processor component 150A further includes one or more controllers 160 and a display 162, collectively referred to as user interface 161. Controller(s) 160 may be any type of controller enabling a person(s) (e.g., the recipient, parent, or clinician) to interface with the sound processor component 150, such as, for example, to adjust one or more parameters of the sound processor component 150, retrieve data from the sound processor component 150, etc. Controller(s) 160 may include, for example, one or more dials, buttons, touchpad(s), keyboard(s). Display 162 may be any type of display, such as an LED or LCD display for displaying information regarding the cochlear implant 100 (e.g., parameters, logged data, etc.).

As will be discussed in more detail below, in other embodiments, the sound processor component 150 may have different configurations. For example, as illustrated in FIG. 1B, the sound processor component 150B may be configured as a behind the ear (BTE) device. In one embodiment, the external component 150B may configured as the BTE device for an adult. In embodiments, in which the cochlear implant 100 is fit to a child and the sound processor component 150B is a BTE, the BTE may initially be placed in a pocket, such as discussed above. Then, when the child grows large enough for the BTE to fit behind the child's ear, the wireless microphone component 170 is no longer used; and the BTE 150B is used to both receive the sound (via a microphone included in the BTE) and generate the stimulation data. If the microphone of the BTE 150B is used for receiving sound 103 when the BTE 150B is placed in a pouch for a child, the sound picked up by this microphone will be the sound arriving at the back of the child, and not the sound arriving at the ear of the child. This may cause spatial perception problems for the child. As such, a small wireless microphone component (i.e., first external component 170), such as discussed above, may be located in or near the child's ear in order to improve the spatial perception of sound by the child.

As illustrated, BTE 150B comprises a fastening device 163 that may configured to connect the BTE 150B to the recipient's clothing. In an embodiment, fastening device 163 may be, for example, a clip. BTE 150B may comprise the same or similar components to the above discussed wireless remote control unit 150A of FIG. 1A. For example, BTE 150B may comprise a primary coil interface 158B such a primary coil interface 158B. Similarly, BTE 150B may comprise a user interface 161 that the recipient may use to adjust one or more parameters (e.g., volume) for the cochlear implant. As noted, user interface 161 may comprise one or more controllers 160 and/or a display 162. Hereinafter, sound processor component 150A and 150B will be referred to simply as sound processor component 150 for ease of explanation of the embodiments of FIGS. 1A and 1B.

Sound processor component 150 may also comprise a power supply 164, such as a battery. This battery may be, for example a rechargeable battery that is either permanently installed in sound processor component 150 or may be removable from sound processor component 150. Or, for example, the battery may be a non-rechargeable replaceable battery.

The internal component 144, which may be temporarily or permanently implanted in the recipient, comprises an internal coil 136 (also referred to herein as secondary coil 136), an implant unit 134, and a stimulating lead assembly 118. As illustrated, implant unit 144 comprises a stimulator unit 120 and a secondary coil interface 132 (also referred to as secondary coil interface 132).

Secondary coil interface 132 is connected to the secondary coil 136. Secondary coil 136 may include a magnet (also not shown) fixed in the middle of secondary coil 136. The secondary coil interface 132 and stimulator unit 120 are hermetically sealed within a biocompatible housing, sometimes collectively referred to as a stimulator/receiver unit. The internal coil receives power and stimulation data from primary coil 130.

Stimulating lead assembly 118, as illustrated, has a proximal end connected to stimulator unit 120, and a distal end implanted in cochlea 140. Stimulating lead assembly 118 extends from stimulator unit 120 to cochlea 140 through mastoid bone 119. In some embodiments stimulating lead assembly 118 may be implanted at least in basal region 116, and sometimes further. For example, stimulating lead assembly 118 may extend towards apical end of cochlea 140, referred to as cochlea apex 147. In certain circumstances, stimulating lead assembly 118 may be inserted into cochlea 140 via a cochleostomy 122. In other circumstances, a cochleostomy may be formed through round window 121, oval window 112, the promontory 123 or through an apical turn 135 of cochlea 140.

Stimulating lead assembly 118 comprises a longitudinally aligned and distally extending array 146 of electrode contacts 148, sometimes referred to as array of electrode contacts 146 herein. Although array of electrode contacts 146 may be disposed on stimulating lead assembly 118, in most practical applications, array of electrode contacts 146 is integrated into stimulating lead assembly 118. As such, array of electrode contacts 146 is referred to herein as being disposed in stimulating lead assembly 118. Stimulator unit 120 generates stimulation signals which are applied by electrode contacts 148 to cochlea 140, thereby stimulating auditory nerve 114. Because, in cochlear implant 100, stimulating lead assembly 118 provides stimulation, stimulating lead assembly 118 is sometimes referred to as a stimulating lead assembly.

In cochlear implant 100, primary coil 130 transfers electrical signals (that is, power and stimulation data) to the internal or secondary coil 136 via an inductive coupled radio frequency (RF) link. Secondary coil 136 is typically made of multiple turns of electrically insulated single-strand or multi-strand platinum or gold wire. The electrical insulation of secondary coil 136 is provided by a biocompatible wire insulator and a flexible silicone molding (not shown). In use, secondary coil 136 may be positioned in a recess of the temporal bone adjacent auricle 110 of the recipient.

In operation sound 103 is received by microphone 172 of wireless microphone component 170. The sound is processed (if applicable) and wireless transmitted via antenna 178 to the sound processor component 150. As noted above, this wireless transmission may be over an electromagnetic radio link (e.g., 2.4 GHz). The transmitted sound is received by antenna 152 and provided to processor 156, which generates data specifying the stimulation to be applied to the recipient. This stimulation data is combined with power from battery 164 and provided to primary coil 130, which transmits the power and data to internal component 144 via a magnetic induction link. The power and data is received by secondary coil 136 and provided to implant unit 134. Implant unit 134 uses the power to power the operations of the implant unit and provide stimulation to the recipient via stimulating lead assembly 118. Implant unit 134 processes the received data to generate the stimulation signals for application of the stimulation.

The above cochlear implant 100 comprising a sound processor component 150, a wireless microphone component 170, and internal component 144 may be configured, for example, as a wireless body area network. For example, as noted above, each of these components may wirelessly communicate with one or more other components of the system 100.

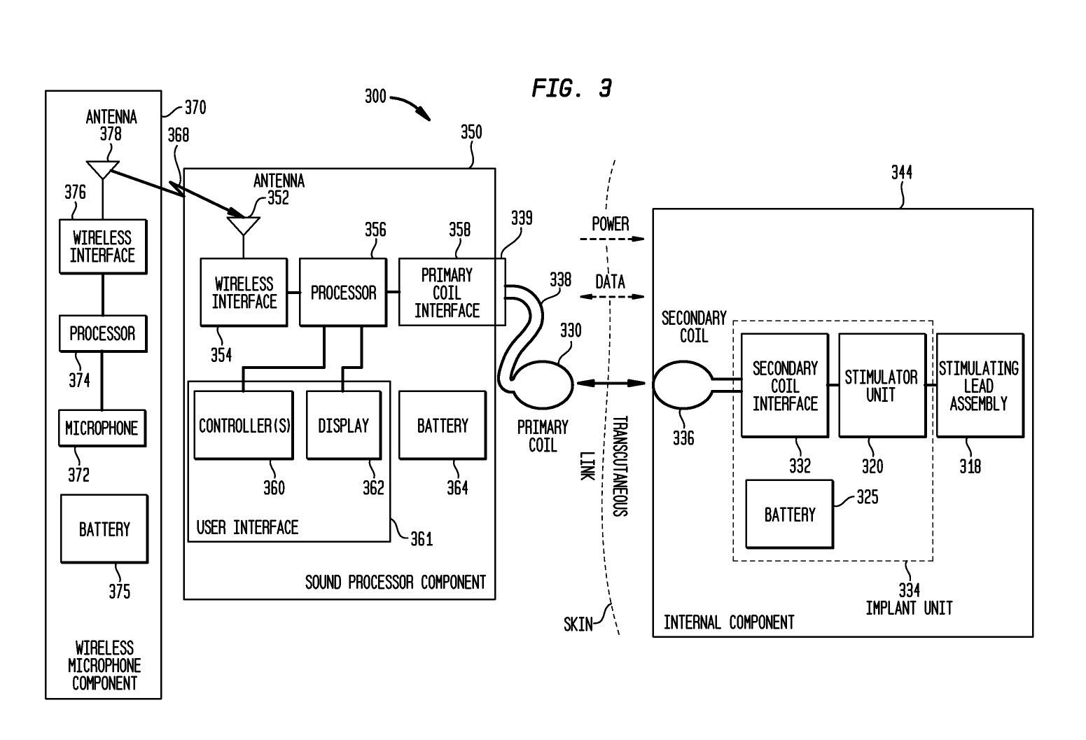

FIG. 3 illustrates an alternative embodiment of a cochlear implant, in accordance with an embodiment. The embodiment may be identical to the above discussed embodiment of FIG. 1 with the exception that implant unit 334 of cochlear implant 300 comprises a power supply 325. Power supply 325 may be, for example, a rechargeable battery for providing power for the operations of implant unit 334.

As illustrated, first external component 370 comprises a microphone 372, a processor 374, a battery 375, a wireless interface 376, and an antenna 378. For ease of explanation, first external component 370 will be hereinafter referred to as wireless microphone component 370. Each of these components may be the same or similar to the similarly named components discussed above with reference to FIGS. 1 and 2.

As shown, second external component 350 (also referred to herein as sound processor component 350) comprises an antenna 352, a wireless interface 354, a processor 356, a primary coil interface 358, a processor 356, a user interface 361, and a power supply 364. Each of these components may be the same or similar to the similarly named components discussed above with reference to FIGS. 1 and 2.

In operation, sound processor component 350 may provide power and data to internal component 344. Implant unit 334 may use the received power to charge battery 325. In the embodiment of FIG. 3, the power and data may be transmitted via magnetic induction (MI) from primary coil 330 to secondary coil 336. Various mechanisms may be used for transmission of the power and data to the internal component 344. For example, in an embodiment, the power and data may be provided simultaneously. Or, for example, a time division multiplexing scheme may be employed, such as, power being transmitted during one time slot and data transmitted during a different time slot.

Or, for example, the internal component 344 may direct when the sound processor component 350 is to transmit data. As an example, in an embodiment, the implant unit may include circuitry for monitoring the power level of the battery 325. If the power of the battery 325 is below a threshold level, the implant unit 344 may send an indication to the sound processor component 350 to transmit power to the internal component 344. If, however, the power level is above the threshold, the implant unit 344 may instruct the sound processor component 350 to not transmit power.

Or, in yet another embodiment, the sound processor component 350 may assess whether the data to be transmitted to the internal component 344 includes useful information (e.g., speech, music, etc.) or not (e.g., noise). If the data includes useful information, the sound processor component 350 may transmit data (but not power) via primary coil 330. While, if the data is deemed to not include useful information, the sound processor component 350 may transmit power (but not data) via primary coil 330.

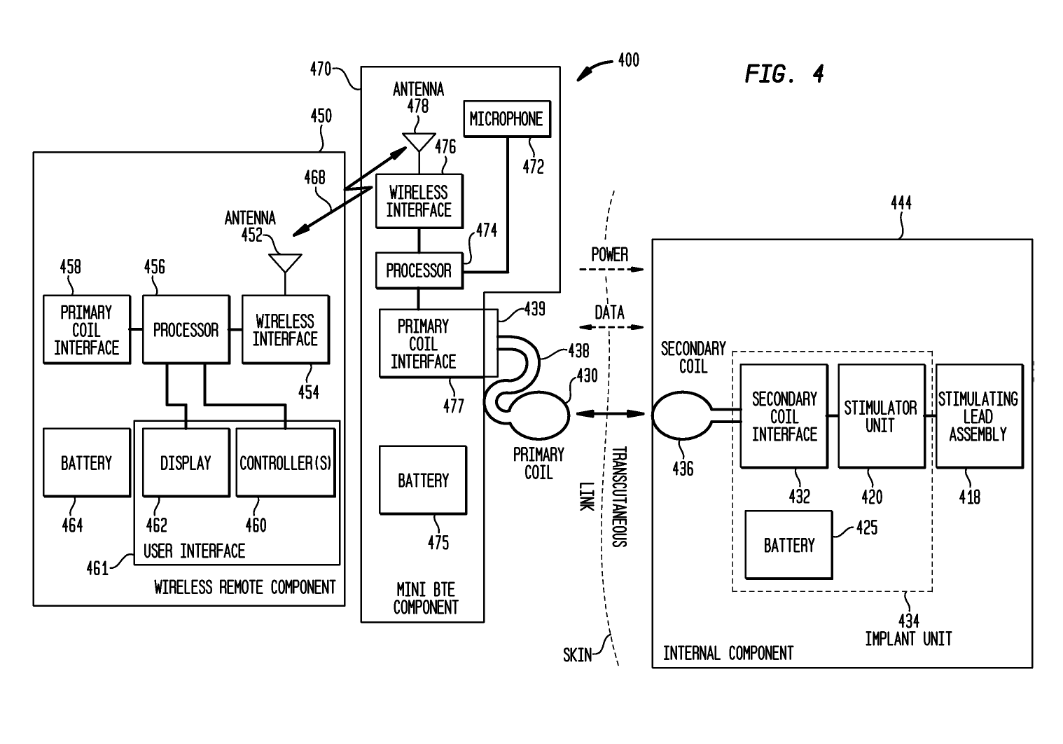

FIG. 4 illustrates an embodiment in which the first external component comprises a primary coil interface similar to the primary coil interface of the second external component, in accordance with an embodiment of the invention. This sound processor component 450 (also referred to herein as wireless remote component 450) of FIG. 4 may be, for example, identical or similar to the sound processor component 350 of FIG. 1A, 1B, or 3. In the illustrated embodiment, sound processor component 450 will be referred to as wireless remote component 450 and may function in a similar manner to the wireless remote component 150A illustrated in FIG. 1A. For example, wireless remote component 450 may be configured to provide power to the internal component 444 when the primary coil interface 458 of the wireless remote component 450 is connected to primary coil 430 via cable 438. In this manner, wireless remote component 450 may be able to charge battery 425 of implant unit 434.

As illustrated, first external component 470 comprises a microphone 472, processor 474, wireless interface 476, and an antenna 478. For ease of explanation, the first external component 470 will be hereinafter referred to as mini-BTE component 470. Each of these components may be the same or similar to the similarly named components of FIG. 1A, 1B, 2 and/or 3.

Mini-BTE component 470 also comprises a primary coil interface 477 in the illustrated embodiment. Primary coil interface 477 may be configured for detachably connecting the wireless microphone component 471 to primary coil 430 via cable 438. For example, cable 438 and primary coil interface 477 may each be configured with a matching connector 439 to connect mini-BTE component 470 to cable 438.

When primary coil interface 477 is connected to cable 438, mini-BTE component 470 may adjust the processing functions performed by processor 474. For example, when primary coil interface 477 is connected to cable 438, processor 474 may process the sound received from microphone 472 to generate data specifying stimulation signals for application of stimulation via stimulating lead assembly 418. This data may be encoded and provided to primary coil 430 for transmission to internal component 444. In an embodiment, processor 474 may process the sound in the same or a similar manner as processor 156 of FIGS. 1-2.

When mini-BTE component 470 is connected to primary coil 430, wireless remote component 450 may function as a wireless remote that a recipient may use to adjust the parameters used by processor 474 in processing the received sound, or, for example, to obtain information from mini-BTE component 470, such as, for example, information regarding the power level of battery 425 of internal component 444 or battery 435 of mini-BTE component 470. When operating as a wireless remote, wireless remote component 450 may wirelessly communicate with mini-BTE component 470 via antennas 452 and 478 in a similar manner as was described above for transmitting wirelessly transmitting sound data over link 168 of FIG. 1.

In the embodiment of FIG. 4, the wireless remote component 450 may be connected to the primary coil 430 in order to charge battery 425 of internal component 444. That is, wireless remote component 450 may provide power and data to internal component 444. Once the battery 425 of the implant unit 434 is charged, the smaller mini-BTE component 470 may be connected to primary coil 430 to provide data (but not power) to internal component 444. When the mini-BTE component 470 is connected to the primary coil interface 430, the internal component 444 may rely on battery 425 for power.

In such an embodiment, the larger wireless remote component 450 may be connected to primary coil 430 to charge battery 425 while the recipient is asleep or expected to be sedentary (e.g., sitting in a chair). Then, the smaller mini-BTE component 470 may be connected to primary coil 430 to process sound and generate stimulation data when the recipient expects to be more active (e.g., when the recipient is awake, playing sports, going for a walk, etc.). In this manner, the recipient may use the larger wireless remote component 450 during periods of rest and use the smaller mini-BTE component 470 during periods of activity.

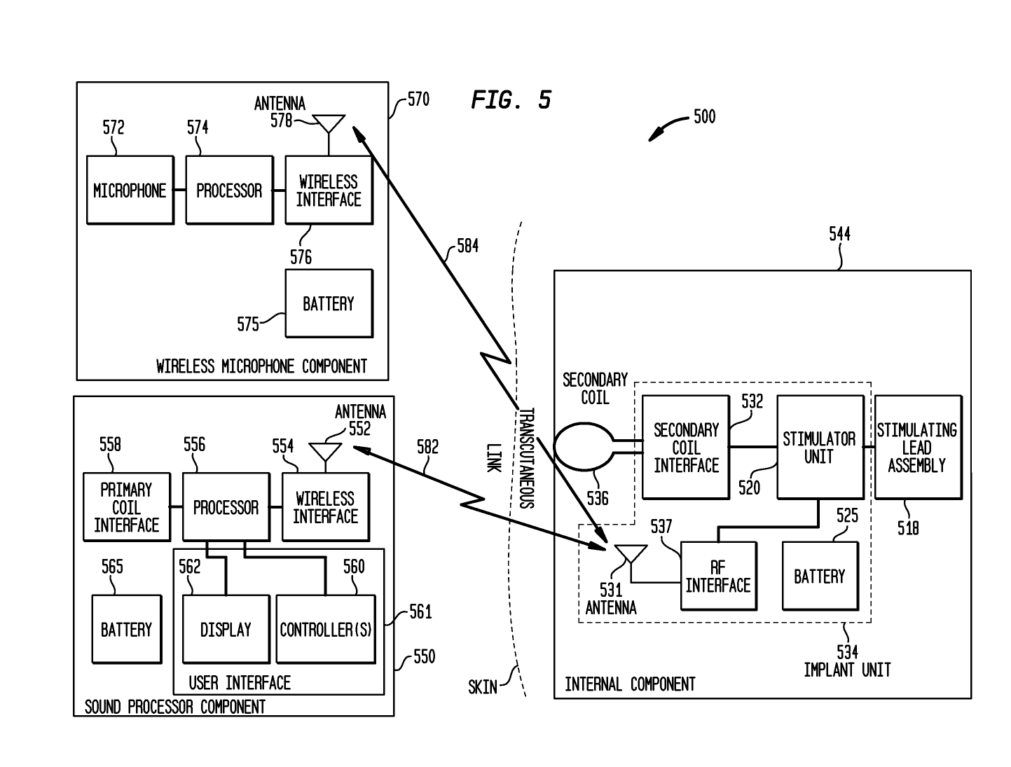

In yet another embodiment, the first and first external components may wirelessly communicate with the internal component using a multiplexing scheme. FIG. 5 illustrates an embodiment in which a second external component 550 and a first external component 570 wirelessly communicate with an internal component 544, in accordance with an embodiment of the present invention. For ease of explanation, the first external component 570 will be hereinafter referred to as wireless microphone component 570 and second external component 550 referred to as sound processor component 550.

In the illustrated embodiment, the sound processor component 570 may comprise the same or similar components as the sound processor component 150 discussed above with reference to FIGS. 1 and 2. Similarly, the wireless microphone component 550 may comprise the same or similar components as the wireless microphone component 170 discussed above with reference to FIGS. 1 and 2. Further, as noted above, the wireless microphone component 570 may have smaller size than that of the sound processor component 550. For example, the wireless microphone component 570 may be configured as an ITE, ITC, CIC, or mini-BTE, or micro-BTE device. The sound processor component 550 may be configured, for example, as a wireless remote control or a standard BTE device.

In the illustrated cochlear implant 500, the first and wireless microphone components 550 and 570, respectively, may wirelessly communicate via communication links 582 and 584, respectively, with the internal component 544 using a multiplexing scheme, such as time division or frequency division multiplexing. For example, in a time division multiplexing scheme, the time domain is divided into fixed length recurrent time slots, one for each communication link 582 and 584, respectively. In a frequency division multiplexing scheme, each communication link 582 and 584 is assigned a different frequency.

In the illustrated embodiment, each communication link may be assigned an RF frequency (e.g., approximately 2.4 GHz) for electromagnetic transmission of information.

As noted, the wireless microphone component 570 may function in a similar manner to wireless microphone component 170 (FIG. 1) or 470 (FIG. 4). For example, sound may be received by microphone 572 and converted to electronic signals that are provided to an optional processor 574. The signal may then be provided to a wireless interface 576 that wireless transmits the signal via antenna 578 to internal component 544 in accordance with the multiplexing scheme employed by system 500.

In the illustrated embodiment, the sound processor component 550 may function as a wireless remote control. For example, a recipient may use the controller(s) 560 and display 562 of sound processor component 550 to adjust parameter(s) and/or obtain information regarding the cochlear implant 500. For example, as shown, sound processor component 550 may wirelessly communicate with the internal component 544 via communication link 582.

In the illustrated embodiment, the wireless microphone component 570 and sound processor component 550, respectively, are configured for electromagnetic wireless communications with the internal component 544. When these devices are wireless communicating with the internal component 544 via communications links 582 and 584, the internal component 544 may rely on its battery 525 for providing power for the internal component 544.

In the illustrated embodiment, the internal battery 525 of the internal component 544 may be recharged by connecting a primary coil to the primary coil interface 558 of the sound processor component 550. Then, when connected the primary coil may be placed within the proximity of the secondary coil 536 (e.g., by aligning the primary and secondary coils as discussed above with reference to FIG. 1). In such a configuration, the wireless microphone component and sound processor component may function in a similar manner as discussed above with reference to FIG. 1.

For example, when the primary and secondary coils are aligned, the processor 556 of the sound processor component 550 may detect this alignment and wireless transmit and indication of this connection via antenna 552. This indication may be received by the internal component 544 (via antenna 531) and the wireless microphone component 570 (via antenna 578). In response, internal component 544 may cease using receiving sound data via antenna 531 and instead may use the secondary coil 536 for receiving power and data from sound processor component 550. Further, in response to this indication, wireless microphone component 570 may begin wirelessly transmitting the sound data to sound processor component 550 via antennas 578 and 552. Thus, after connecting the sound processor component 550 and internal component 544 via a primary coil and secondary coil 536, the cochlear implant 500 may function similar to cochlear implant 100 of FIG. 1.

Although the above embodiment of FIG. 5 was discussed with reference to electromagnetic transmissions between the wireless microphone component 570, the sound processor component 550 and the internal component 544, it should be noted that in other embodiments, these wireless communications may be via magnetic induction and the antennas 578 and 552 may be coils or other devices for data transfer via magnetic induction. In such an embodiment, internal component 544 may not include a separate antenna 531, but instead use secondary coil 536 for receipt of the power and data information. Similarly, in such an embodiment, the wireless microphone component 570 and sound processor component 550 may communicate with the internal component 544 for data transfer using a multiplexing scheme. Then, when sound processor component 550 transmits power to internal component 544 for charging battery 525, the sound processor component 550 may transmit and indication that is received by the wireless microphone component 570. In response, wireless microphone component 570 transmits the sound data to sound processor component 550, which combines the sound data with the power data transmitted to the internal component 544.

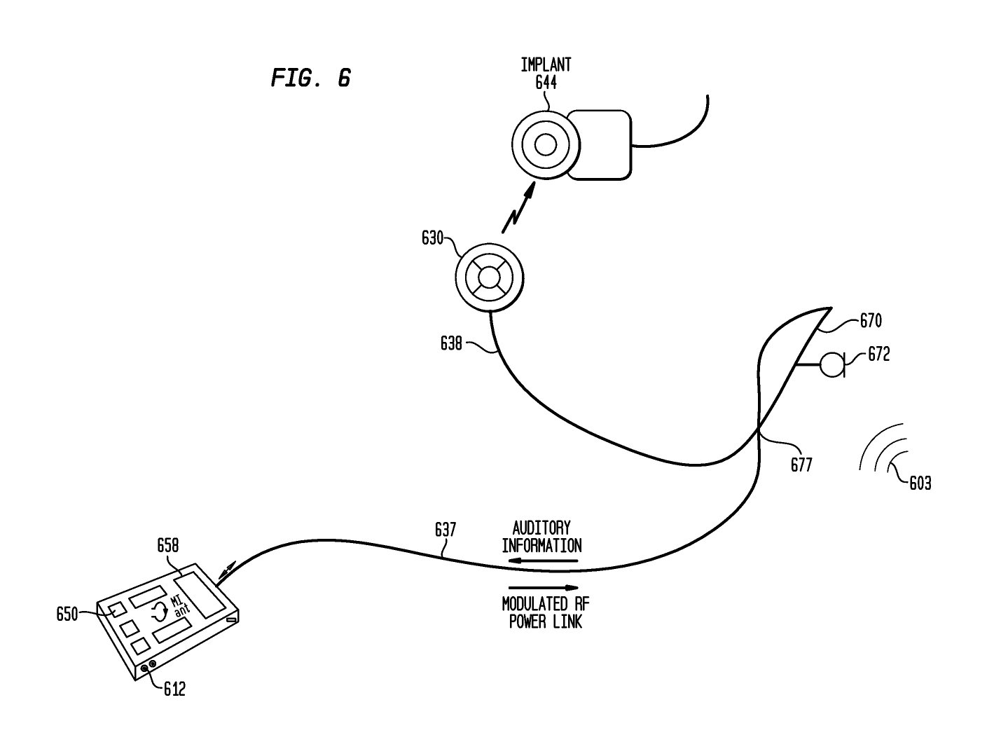

FIG. 6 illustrates a cochlear implant comprising first and second external components and an internal component, where the wireless microphone component supports part of the weight of the cable connected to the second external component, in accordance with an embodiment of the invention. For ease of explanation, the first external component 670 will be hereinafter referred to as wireless microphone component 670 and second external component 650 referred to as sound processor component 650.

In the illustrated embodiment, the wireless microphone component 670 may be the same or similar to the above-discussed wireless microphone components of FIGS. 1-5. In this example, the wireless microphone component is configured as a micro BTE that may fit behind the ear of the recipient. However, in other embodiments, the wireless microphone component 670 may have different configurations, such as, for example, a mini BTE with retain mechanism.

As shown, the wireless microphone component 670 is connected to a cable 637 connected to the sound processor component 650 and a cable 638 connected to the primary coil 630. Each of these cables 630 and 638 may be detachably connected to an interface 677 of the wireless microphone component 670.

In use, the wireless microphone component 670 may be attached (or positioned in) to a portion of the recipient (e.g., behind the ear) or the recipient's clothing such that the wireless microphone component 670 may support at least a portion of the weight of cables 637 and 638. For example, if the primary coil 630 is connected directly to the sound processor component 550 and located far from the primary coil 630, the length of the cable connecting the primary coil 630 and sound processor component 650 may be large, such as, for example, in an embodiment in which the sound processor component 650 is a wireless remote for a child that is configured to be placed in a pocket in the clothing on the back of the child. In such a situation, the primary coil 630 may need to support the majority of the weight of the cable. This may result in the primary coil 630 more readily becoming detached from the recipient or to lose alignment with the implanted secondary coil of the internal component 644.

Using an embodiment such as illustrated in FIG. 6 allows the wireless microphone component 670 to reduce the length of cable connected from the primary coil 630 and to support at least part of the weight of this cable 638. This may result in the primary coil 630 being less likely to lose alignment with the implanted secondary coil.

In operation, sound 603 may be received by the microphone of the wireless microphone component 670. This sound may be transmitted to the sound processor component 650 via cable 637. The sound processor component 650 may then process the sound and generate stimulation data specifying the stimulation to be applied to the recipient. The sound processor component 650 may then forward this stimulation data and/or power to the wireless microphone component 670 via cable 638. Via the wireless microphone component 670 the stimulation data and/or power is passed to the primary coil 630 via cable 638. The primary coil 630 then transfers the data and/or power to the internal component 644 via magnetic induction transcutaneously.

In an embodiment, in which the internal component 644 comprises a rechargeable battery, the sound processor component 650 may be disconnected form the wireless microphone component 670 (that is cable 637 may be disconnected from interface 677) and the internal component 644 may rely on its internal battery for power. Further, in such an embodiment, the wireless microphone component 670 may include a processor configured to process the sound received by the microphone 672 and generate the stimulation data for application of stimulation.

Further, in an embodiment, the primary coil 630 may be disconnected from the wireless microphone component 670 (i.e., cable 638 disconnected from interface 677). Once disconnected, the second internal component 650 may wirelessly transmit stimulation data to the internal component 644. For example, wireless microphone component 670 may include an RF wireless interface and antenna for wireless sending stimulation data to the internal component 644.

Or, for example, wireless microphone component 670 may comprise an internal primary coil that it may use to wireless transmit the stimulation data to the secondary coil of the internal component 644. Because the wireless microphone component transmits the stimulation data via magnetic induction in this example, it may be beneficial to ensure the wireless microphone component 670 is within a specified distance (e.g., less than 10 cm) of the secondary coil of the internal component 644.

As shown, sound processor component 650 further comprises an auxiliary input 612 that may be used to connect the sound processor component 650 to another device, such as an MP3 player, cell-phone, or other device configured to provide audio signal(s). When a device is connected to the auxiliary input 612, the sound processor component 650 may generate stimulation data in accordance with the audio signal(s) received via the auxiliary input 612. In operation, the sound processor component 650 may be configured to only process the audio signal(s) receive via the auxiliary input 612 when a device is connected to the auxiliary input 612. Or, for example, the sound processor component 650 may generate stimulation data for both signals received from the wireless microphone component 670 and via the auxiliary input 612. Further, the sound processor component 650 may include or more controller(s) that a user may use to adjust what stimulation data is generated. That is, a user may use the controller(s) to select whether signals from the auxiliary input 612, from the wireless microphone component 670, or both, are processed in generating the stimulation. Further, if both are processed, the controller(s) may enable the recipient to adjust one or more parameters regarding how they are processed, such, as the individual gain applied to each signal.

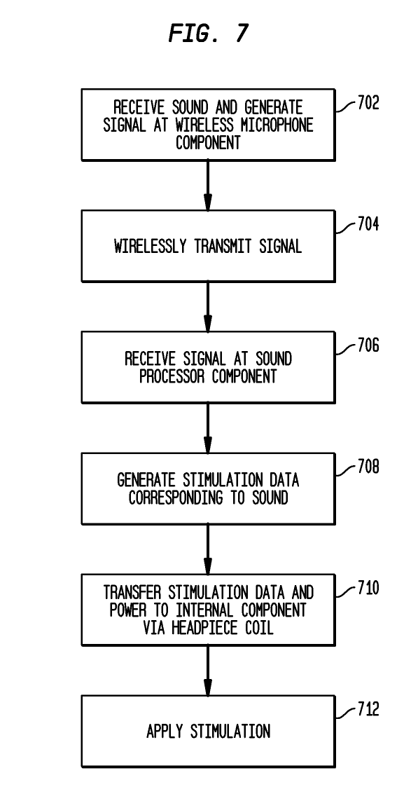

FIG. 7 provides a simplified flow chart for receiving sound and providing corresponding stimulation, in accordance with an embodiment of the present invention. FIG. 7 will be discussed with reference to the above-discussed FIGS. 1 and 2.

At block 702, sound 103 is received by microphone 172 of wireless microphone component 170 and converted to an electronic signal. At block 704, the electronic signal is optionally processed by processor 174 and wirelessly transmitted by wireless interface 176 via antenna 178. The sound processor component 150 then receives the transmitted signal via antenna 152 and wireless interface 154 at block 706. Processor 156 then processes the received signal to generate stimulation data at block 708. Primary coil interface 158 of the sound processor component 150 then transcutaneously transmits, at block 710, the stimulation data along with power via primary coil 130. Secondary coil interface 132 of the internal component 144 then receives the power and stimulation data via secondary coil 136 and provides the stimulation data to stimulator unit 120, which applies corresponding stimulation to the recipient via stimulating lead assembly 118, at block 712.

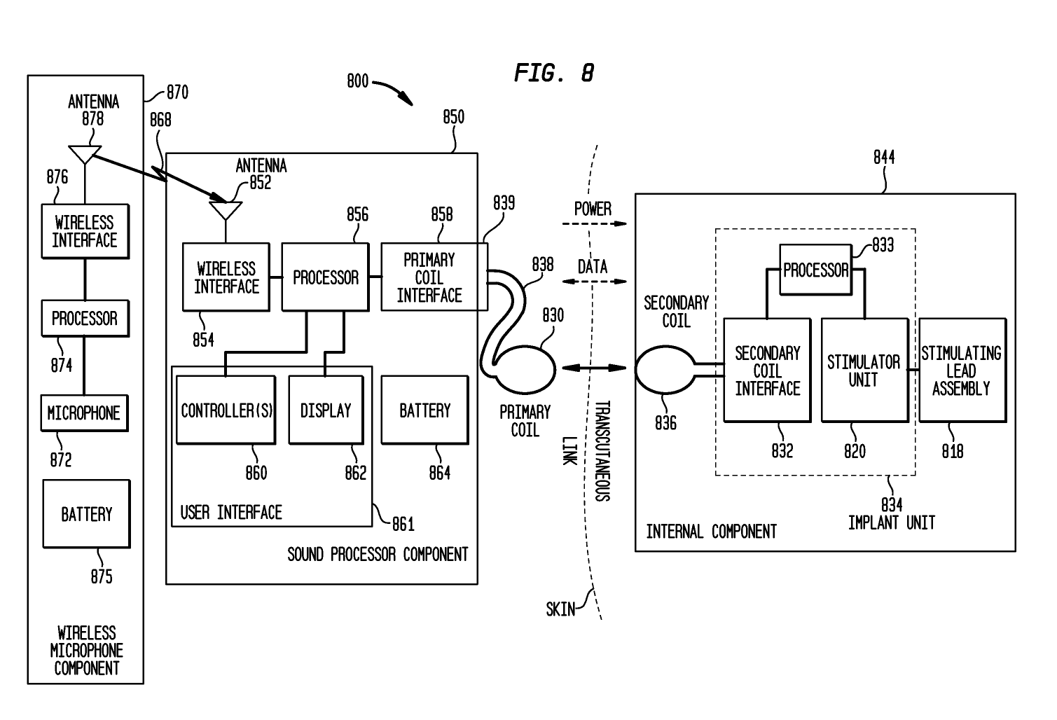

FIG. 8 illustrates an alternative embodiment of a cochlear implant, in accordance with an embodiment. The embodiment may be identical to the above discussed embodiment of FIG. 1 with the exception that implant unit 834 of cochlear implant 800 comprises a processor 833 that may, for example, perform the same or similar functionality to that of processor 856 of the sound processor component 850.

As illustrated, wireless microphone component 870 comprises a microphone 872, a processor 874, a battery 875, a wireless interface 876, and an antenna 878. Each of these components may be the same or similar to the similarly named components discussed above with reference to FIGS. 1 and 2.

Further, as shown, second external component 850 (also referred to herein as sound processor component 850) comprises an antenna 852, a wireless interface 854, a processor 856, a primary coil interface 858, a processor 856, a user interface 861, and a power supply 864. Each of these components may be the same or similar to the similarly named components discussed above with reference to FIGS. 1 and 2.

In such an example, when primary coil interface 858 is connected to primary coil 830, processor 856 of sound processor component 850 may provide minimal processing of sound from wireless microphone component 870. Rather, sound processor component 850 may provide the electronic signals representative of the received sound to the internal component 844.

Internal component 844 may then receive these electronic signals and provide the received signals to processor 833, which generates the stimulation data. As noted above, the stimulation data specifying the stimulation to be applied to recipient. Other than the operation of processor 833, the components of internal component 844 may function in a similar manner to the similarly named components discussed above with reference to FIGS. 1 and 2.

When sound processor component 850 is not connected to the primary coil, wireless microphone 870 may wirelessly transmit electronic signals corresponding to received sound to internal component 844. In such an embodiment, antenna 878 may be, for example, an antenna for RF transmission or a coil for transmission via magnetic induction.

If wireless component 870 transmits the signals via magnetic induction, internal component 844 may receive and provide these signals to processor 833 via secondary coil 836 and secondary coil interface 832. If, however, wireless microphone component 870 transmits the signals via RF, internal component may include an RF antenna and RF interface, such as discussed above with reference to FIG. 5, that provides the received signals to processor 833.

In use, sound processor component 850 may be used to provide power and/or data (e.g., electronic signals representative of sound) to internal component 844 for recharging battery 825, such as discussed above with reference to FIG. 3. When battery 825 is charged, the smaller microphone component 870 may provide electronic signals representative of received sound to internal component 844. Because processor 833 of internal component 844 may generate the stimulation data for the cochlear implant 800 for the received sound, wireless microphone 870 may be a small simple device that, for example, does not include processor 874.

Embodiments of the present invention have been described with reference to several aspects of the present invention. It would be appreciated that embodiments described in the context of one aspect may be used in other aspects without departing from the scope of the present invention.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

1. A hearing prosthesis system comprising:
an implantable component configured to deliver stimulation to a recipient of the hearing prosthesis system;
a first smaller external component comprising:
at least one sound input element configured to be located on, near, or in a recipient's ear and configured to receive sound and generate an electronic signal based on the received sound; and
a first wireless interface configured to wirelessly transmit the electronic signal;
a second larger external component comprising:
a second wireless interface configured to receive the transmitted electronic signal;
a processor configured to process the received electronic signal and generate corresponding stimulation data that specifies stimulation;
a coil interface configured to transfer the stimulation data;
a power supply; and
a user interface;
a first coil system comprising a first coil, a cable and a connector, wherein the connector is detachably connectable to the coil interface of the second external component, wherein the cable is configured to enable the second external component to be positioned, when the cable connects the second external component and the first coil, at a location remote from the first external component, and wherein the first coil is configured to transcutaneously transfer the stimulation data to the implantable component.

2. The hearing prosthesis system of claim 1, wherein the coil interface of the second external component is further configured to transcutaneously transfer power from the power supply via the first coil.

3. The hearing prosthesis system of claim 2, wherein the implantable component comprises:
at least one electrode contact configured to apply stimulation to the recipient;
a second coil configured to receive the power and the stimulation data; and
a rechargeable battery configured to be recharged using the received power transcutnaneously transferred by of the first coil.

4. The hearing prosthesis system of claim 1, wherein the first coil and second coil each comprise a magnet for alignment of the first and second coils.

5. The hearing prosthesis system of claim 1, wherein the first external component is configured as an in-the-ear (ITE) device, an in-the-canal (ITC) device, a completely in canal (CIC) device, a mini-Behind the Ear (BTE) device, or a micro-BTE.

6. The hearing prosthesis system of claim 1, wherein second external component is configured to enable a recipient to adjust one or more parameters of the hearing prosthesis.

7. The hearing prosthesis system of claim 6, wherein the second external component comprises:
at least one controller configured for receiving information from the recipient; and
a display configured to display information.

8. The hearing prosthesis system of claim 1, wherein the second external component is configured as a Behind the Ear (BTE) device.

9. The hearing prosthesis system of claim 1, wherein the second external component has wireless remote control functionality with the first external component or implantable component when the first coil is detached from the second external component.

10. The hearing prosthesis system of claim 1, wherein the implantable component is an implantable components for at least one of a cochlear implant, a DACS implant, and a TBAHA implant.

11. A method comprising:
receiving sound at a first external component;
generating an electronic signal based on the received sound;
wirelessly transmitting the electronic signal from the first external component to a second external component, where the first external component is located on, near, or in a recipient's ear;
generating, at the second external component, data corresponding to the received sound;
providing the data via a cable to an external coil of an inductive link with an internal component;
transferring the data to the internal component transcutaneously over the inductive link; and
applying stimulation to a recipient in accordance with the transmitted data.

12. The method of claim 11, further comprising:
transferring transcutaneously power from the second external component to the internal component over a wireless radio frequency (RF) link.

13. The method of claim 12, further comprising:
recharging a rechargeable battery of the internal component by power transferred transcutaneously over the wireless link.

14. The method of claim 11, wherein the first external component is configured as an in-the-ear (ITE) device, an in-the-canal (ITC) device, a completely in canal (CIC) device, a mini-Behind the Ear (BTE) device, or a micro-BTE.

15. The method of claim 11, further comprising:
adjusting, by a recipient using the second external component, one or more parameters of the hearing prosthesis.

16. The method of claim 11, wherein the second external component is configured as a Behind the Ear (BTE) device.

17. The method of claim 11, further comprising:
detachably connecting the first external component to a first coil.

18. The method of claim 11, further comprising:
detachably connecting the second external component to a first coil.

19. The method of claim 11, wherein the internal component is an internal component for at least one of a cochlear implant, a DACS implant, and a TBAHA implant.

20. The method of claim 11, further comprising:
generating stimulation data based on the received sound, wherein the stimulation data specifies stimulation to be applied to the recipient; and
the data transferred to the internal component transcutaneously over the inductive link is the generated stimulation data.

21. The method of claim 11, further comprising:
generating stimulation data in the internal component based on the data transferred to the internal component, wherein the stimulation data specifies stimulation to be applied to the recipient.

22. A hearing prosthesis system comprising:
means for receiving sound at a first external component;
means for generating an electronic signal based on the received sound;
means for wirelessly transmitting the electronic signal from the first external component to a second external component, where the first external component has a smaller size than the second external component and is configured to be located on, near or in a recipient's ear;
means for transferring data corresponding to the received sound to an internal component transcutaneouly over a wireless link;
means for detachably connecting the second external component to a cable configured to connecting the second external component and the means for transferring, wherein the cable is configured to enable the second external component to be positioned, when the cable connects the second external component and the means for transferring, at a location remote from the first external component;
means for transcutaneously receiving the stimulation data; and
means for applying stimulation to a recipient in accordance with the stimulation data.

23. The method of claim 11, wherein:
the first external component has a smaller size than the second external component.
